(19) United States
(12) Patent Application Publication
(10) Pub. No.: US 2016/0374904 A1
(43) Pub. Date: Dec. 29, 2016

(54) Sequential Material Deposition for Remineralization and Desensitization of Teeth

(71) Applicant: South Dakota Board of Regents, Pierre, SD (US)

(72) Inventor: Grigoriy Sereda, Vermillion, SD (US)

(21) Appl. No.: 15/189,228

(22) Filed: Jun. 22, 2016

Related U.S. Application Data
(60) Provisional application No. 62/183,220, filed on Jun. 23, 2015.

(51) Int. Cl. A61K 6/00; A61K 8/58; A61K 8/19; A61K 8/73; A61K 8/24; A61C 15/04; A61K 8/34; A61K 8/365; A61K 8/29; A61K 8/26; A61C 15/02; A61Q 11/00; A61K 8/72

(52) U.S. Cl. CPC A61K 6/0047; A61Q 11/00; A61K 6/007; A61K 6/0008; A61K 8/585; A61K 8/19; A61K 8/736; A61K 8/733; A61K 8/24; A61K 8/72; A61K 8/347; A61K 8/365; A61K 8/29; A61K 8/26; A61C 15/02; A61C 15/041; A61K 2800/92; A61K 2800/592; A61K 2800/28; A61K 2800/74

(57) ABSTRACT

The present invention relates to a method of remineralizing and desensitizing teeth utilizing the sequential steps of first applying to the teeth a first component that attaches to the teeth and secondly applying to the teeth treated with the first component a second component that attaches to the first component.

### CROSS REFERENCE TO RELATED APPLICATIONS

This application claims priority from U.S. Provisional Application Ser. No. 62/183,220, filed Jun. 23, 2015.

### FIELD OF THE INVENTION

The present invention relates generally to dental health and, more specifically, to a method of applying to exposed dentinal tubules two or more agents that act in a synergistic fashion to occlude the open tubules and decrease dental sensitivity and to serve as a scaffold for remineralization.

### BACKGROUND OF THE INVENTION

Demineralization of the teeth leads to hypersensitivity and dental caries, both significant public health concerns. Demineralization is primarily caused by acids present in the mouth, whether through the intake of acidic foods or beverages, the production of acids by bacteria present in the mouth, or the regurgitation of stomach acids into the mouth. Demineralization exposes the dentinal tubules allowing access to dental nerves. It also weakens the hard surfaces of the teeth, increasing the incidence of dental caries.

There are currently three materials on the market that are proved to facilitate tooth remineralization by supplying hydroxyapatite to the tooth surface. The first material is Casein PhosphoProtein Amorphous Calcium Phosphate (CPP-ACP, Recaldent®), which is the subject of an issued US patent by an Australian professor Eric Reynolds'. The active component is a synthetic complex of amorphous hydroxyapatite with a phosphoprotein that binds both the amorphous mineral and the tooth surface with the serine phosphate residues in the protein's sequence. This complex is naturally present in mammals' milk and has shown to promote tooth remineralization as a component of Ml Paste², chewing gum³, mouth rinse³, and sealants³, and when added to bovine milk^{3,4}. In addition, CPP-ACP decreases tooth hypersensitivity by occluding the dentinal tubules and protecting the tooth's pulp chamber from penetration by saliva and food components⁵.

The second material NovaMin® was patented in 2010⁶ as the remineralizing⁷ and desensitizing⁸ component of Dr. Collin's Restore® toothpaste. This material is described as sodium calcium phosphosilicate of unspecified chemical structure and composition⁶. Despite the lack of the proper description of NovaMin®, one can guess that it consists of some kind of inorganic particles containing ionic components of hydroxyapatite, which somehow bind to the tooth surface.

The natural ability of hydroxyapatite nanoparticles to blend with the tooth surface was utilized in the third material, Biorepair®, and similar desensitizing and remineralizing toothpastes⁹. Currently the leading desensitizing product available commercially is the toothpaste Sensodyne®, which delivers its desensitizing effect by the hydrolysis of a relatively toxic active component, tin diftuoride.

All existing dentifrices are based on the simultaneous application of active components to the tooth surface. The active components are often either non-natural materials (silica-based), or require the presence of parabene preservatives, unnecessarily exposing the consumer to potentially harmful chemicals.

The present invention makes use of two components that act in a synergistic fashion, utilizing their chemical or physical interactions to result in crosslinking, specific layer-by-layer deposition of components, or a combination of both cross-linking and layer-by-layer deposition. The components lead to occlusion of the tubules both relieving sensitivity and serving as a scaffold for remineralization. The components can be supplied in foods, dentures, retainers, or mouth guards rather than the classic dentifrices and mouthwashes commonly used for maintenance of dental hygiene. The components can be supplied by either different dentifrices (such as mouthwash and toothpaste), or different domains of the same dentifrice (such as different layers of a chewing gum, or microcapsules embedded into a chewing gum), or by saliva. In the last example, the energy of chewing is harnessed to mix the components and initiate their layer-by-layer deposition, cross-linking, or both.

While the idea of using at least two components and a chemical reaction to occlude dentinal tubules is not entirely new (see, e.g., U.S. Pat. Nos. 9,040,025; 9,011,823; 8,557,224; 8,877,165; 8,865,135; 8,834,850; 5,766,328; 6,506,055; 6,406,529; 5,879,663), the present invention is not obvious in view of what is described in the existing art, and none of the referenced patents describe a remineralization strategy or sequential deposition of the components.

The idea of fortifying edibles and chewing gums with components necessary to maintain dental hygiene is also not new (see, e.g., U.S. Pat. No. 8,524,198 as well as relevant scholarly works²⁻⁵), though the idea of pairing foods to attain the described sequential deposition is a non-obvious advance in the art.

### BRIEF DESCRIPTION OF THE DRAWINGS

FIG. 1 shows occlusion of dentinal tubules by carboxylated silica nanoparticles. The adhesion achieved by this method is resistant toward challenge with 30 seconds of ultrasound.

FIG. 2 shows occlusion of dentinal tubules by the sequential deposition of chitosan and alginate. The adhesion achieved by this method is resistant toward challenge with 30 seconds of ultrasound.

FIG. 3 shows no adhesion of Crest® to dentin.

FIG. 4 shows adhesion of Crest® to dentin pre-treated with chondroitin. The adhesion achieved by this method is resistant toward challenge with 30 seconds of ultrasound.

FIG. 5 shows little occlusion of dentinal tubules by 1% arginate.

FIG. 6 shows significant occlusion of dentinal tubules by 1% arginate followed by treatment with calcium ions. The adhesion achieved by this method is resistant toward challenge with 30 seconds of ultrasound.

FIG. 7 shows occlusion by the sequential treatment by 1% chitosan and 10% sodium citrate. The adhesion achieved by this method is resistant toward challenge with 30 seconds of ultrasound.

FIG. 8 shows no adhesion of bentonite to dentin.

FIG. 9 shows adhesion of bentonite to dentin pretreated with calcium ions. The adhesion achieved by this method is resistant toward challenge with 30 seconds of ultrasound.

### SUMMARY OF THE INVENTION

The subject of the invention is delivery of remineralizing, desensitizing, and other functional components to the tooth surface by the sequential deposition of oppositely charged (or non-electrostatically interacting) components on the tooth surface, especially inside dentinal tubules. Those components include nanoparticles, microparticles, polymer films, phospholipids, and small molecules, pristine or modified by remineralizing, desensitizing, antibacterial, fluoride-supplying, and other functional groups. The components are contained in different dentifrices (toothpaste, mouth rinse, chewing gum, dental floss, toothpicks, etc.), dentures, retainers, mouth guards, or foods, and are deposited on the tooth surface by sequential application of the listed dentifrices, consumption of foods, or combinations of thereof. The positively charged components may include chitosan, polylysine, arginine, calcium cations, proteins, functionalized nano- and micro-particles, and polymer films. The negatively charged components may include bare and functionalized silica particles, chondroitin, hyaluronic acid, alginate, nucleic acids, citrates, and polymer films. The neutral components may include phospholipids, particles of calcium citrate, and particles of hydroxyapatite. The components form the functional material at the tooth surface and inside the dentinal tubules by the mechanism of layer-by-layer deposition, cross-linking, or combinations of both. In certain instances, the energy of chewing can be harnessed to mix the components and initiate their layer-by-layer deposition, cross-linking, or both.

The new methods enable the synergistic effect of desensitizing and remineralization due to the known effect of guided remineralization of hydroxyapatite on matrixes of biopolymers. Thus, deposition of hydroxyapatite on chitosan-chondroitin matrixes is one of the chief foci of bone tissue bioengineering¹⁰.

It is an object of the present invention to prevent or ameliorate the incidence of dental caries and treat hypersensitivity of the teeth, thereby improving the quality of life of subjects and reducing health care costs.

Another object of the present invention is to provide products which can be used not only by health care providers when treating patients but also by subjects on their own.

A further object of the invention is to open up an entirely new market of tooth-healing foods.

### DETAILED DESCRIPTION OF PREFERRED EMBODIMENTS

The approach of the present invention of sequential application utilizes advantages of the layer-by-layer deposition of material chemically or physically bound to one another. This methodology will create a robust desensitizing seal inside the dentin tubules. Since the deposited materials can be imparted with additional desensitizing, antibacterial, remineralizing, and drug-delivering (such as fluoride) properties, the proposed technique is significant improvement versus existing dentifrice materials. In addition, the capability of multilayer films to deliver functional materials (fluoride, calcium, potassium, eugenol, etc.) to the tooth surface and saliva in a time-controlled fashion is especially critical for the prolonged periods between meals when the natural saliva (stationary saliva) flow is the lowest, least alkaline, and minimally protective to the tooth. The employed components can be selected from a wide range of non-toxic natural materials such as chondroitin, calcium citrate, chitosan, and strawberry DNA. A significant innovation of the new method is utilizing the synergistic effect of desensitization and remineralization due to the guided deposition of hydroxyapatite on the scaffolds formed by the desensitizing agents in the process of dentinal tubule occlusion. Utilization of a food-grade alginate cross-linked by calcium ions, may allow for the use of culinary recipes instead of medicated compositions. The term “dentifrice” as used throughout this description, denotes a paste, gel, or liquid formulation. The dentifrice may be in any desired form, such as toothpaste; (including deep striped, surface striped, multi-layered, having a gel surround the paste); powder; beads; mouthwash; mouth rinses; lozenge; dental gel; a periodontal gel; a liquid suitable for painting a dental surface; a chewing gum; a dissolvable, partially dissolvable or non-dissolvable film or strip; a wafer; a wipe or towelette; an implant; a foam; a troche; a dental floss, liquid formulated for oral application in a small portable nebulizer (spray bottle), liquid formulated for oral application in a small portable drop-generating, bottle, a soft pliable tablet (“chewie”), or any combinations thereof.

The invention will now be described with reference to the following non-limiting examples.

### EXAMPLE 1

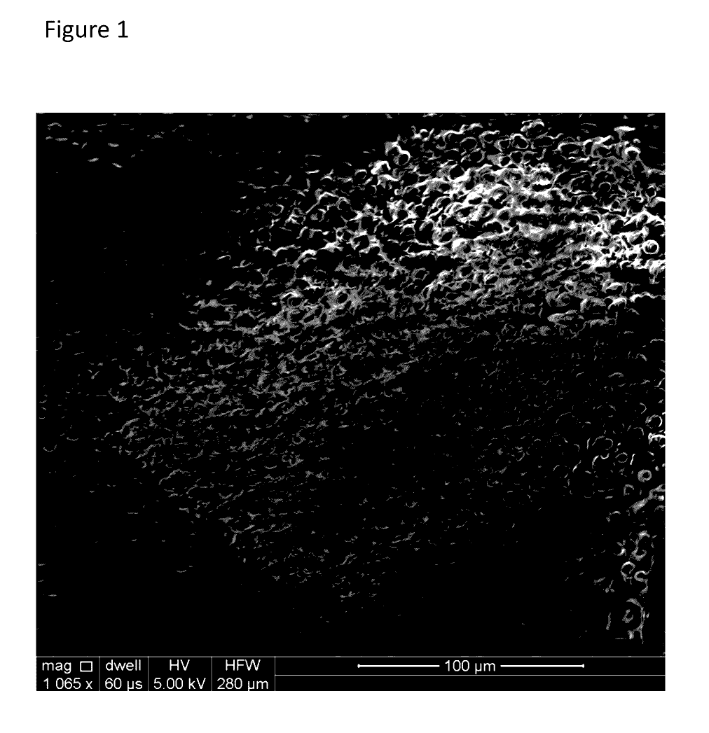

Application 1: Toothpaste containing potassium carboxylate-functionalized silica nanoparticles that, due to the presence of carboxy-groups, bind to the tooth surface occluding the tubules and additionally delivering the desensitizing material (potassium cation) to the tooth. See FIG. 1.

Application 2: Mouth rinse containing antibacterial chitosan nanoparticles (positively charged) that cover the already deposited silica nanoparticles (negatively charged) and seal the tubules enhancing the desensitizing effect.

### EXAMPLE 2

Application 1: Mouth rinse containing antibacterial chitosan (positively charged) that electrostatically interacts with dentin.

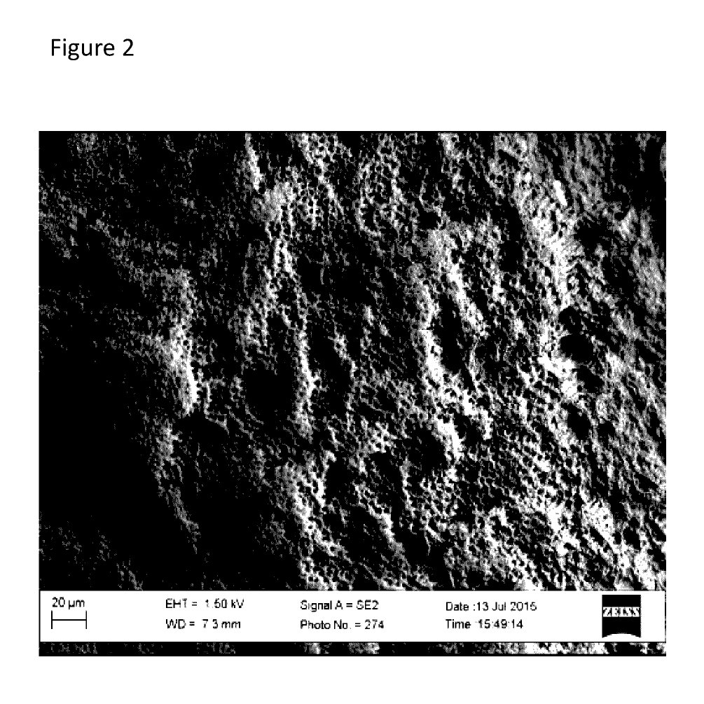

Application 2: Mouth rinse containing alginate (negatively charged) that covers the already deposited chitosan (positively charged) and seals the tubules enhancing the desensitizing effect. The deposited chitosan provides a site for remineralization clue to its affinity to calcium ions. See FIG. 2.

### EXAMPLE 3

Application 1: Toothpaste containing nanocrystals of hydroxyapatite coated with chondroitin sulfate that bind to the tooth surface due to the presence of carboxy-groups in chondroitin, and deliver the remineralizing material (hydroxyapatite) to the tooth.

Application 2: Mouth rinse containing antibacterial chitosan nanoparticles (positively charged) that cover the already deposited chondroitin (negatively charged) and seal the tubules enhancing the desensitizing effect. In addition, the chitosan-chondroitin scaffold will promote remineralization by hydroxyapatite and its components from saliva.

Application 3: Dental floss or toothpicks functionalized by hydroxyapatite or calcium citrate will provide additional material for remineralization.

### EXAMPLE 4

Application 1: Toothpaste containing nanocrystals of hydroxyapatite coated with DNA, including without limitation strawberry DNA, that bind to the tooth surface due to the presence of phosphate-groups in the DNA and deliver the remineralizing material (hydroxyapatite) to the tooth.

Application 2: Dental floss modified with eugenol-loaded antibacterial chitosan nanoparticles (positively charged) that cover the already deposited DNA (negatively charged) and seal the tubules enhancing the desensitizing effect, which is additionally enforced by eugenol.

### EXAMPLE 5

Application 1: Toothpaste containing nanocrystals of calcium citrate grown in the presence of chondroitin sulfate that bind to the tooth surface due to the presence of carboxy-groups in chondroitin, and deliver the remineralizing material (calcium) to the tooth.

Application 2: Mouth rinse containing antibacterial chitosan nanoparticles (positively charged) that cover the already deposited chondroitin (negatively charged) and seal the tubules enhancing the desensitizing effect. In addition, the chitosan-chondroitin scaffold will promote remineralization by hydroxyapatite and its components from saliva.

Application 3: Dental floss or toothpicks functionalized by hydroxyapatite or calcium citrate will provide additional material for remineralization.

### EXAMPLE 6

Application 1: Mouth rinse containing potassium chondroitin sulfate that binds to the tooth surface due to the presence of carboxy-groups in chondroitin, and delivers the desensitizing material (potassium) to the tooth.

Application 2: Mouth rinse containing antibacterial chitosan fluoride nanoparticles (positively charged) that cover the already deposited chondroitin (negatively charged), seal the tubules enhancing the desensitizing effect, and deliver fluoride strengthening the tooth. In addition, the chitosan-chondroitin scaffold will promote remineralization by hydroxyapatite and its components from saliva.

Application 3: Dental floss or toothpicks functionalized by hydroxyapatite or calcium citrate will provide additional material for remineralization.

### EXAMPLE 7

Application 1: Mouth rinse containing potassium chondroitin sulfate that binds to the tooth surface due to the presence of carboxy-groups in chondroitin, and delivers the desensitizing material (potassium) to the tooth.

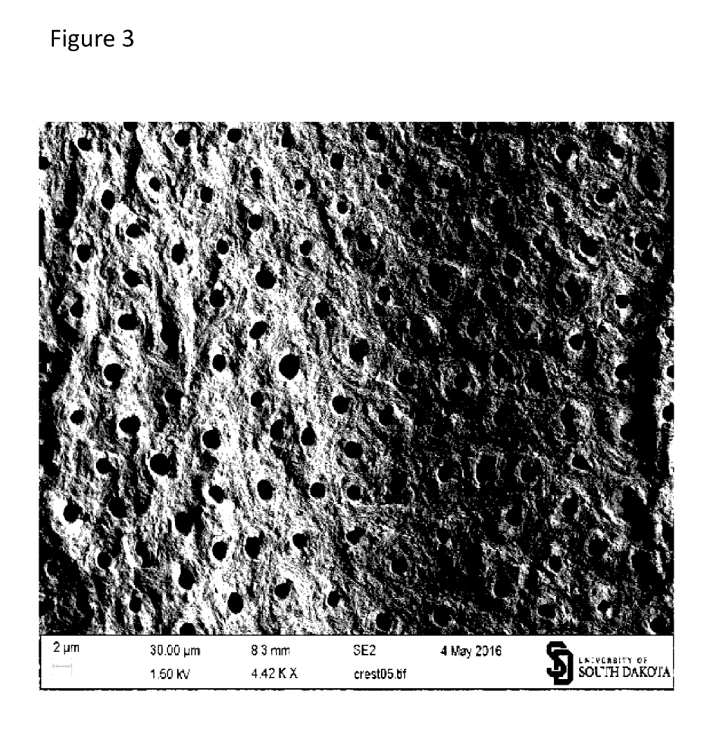

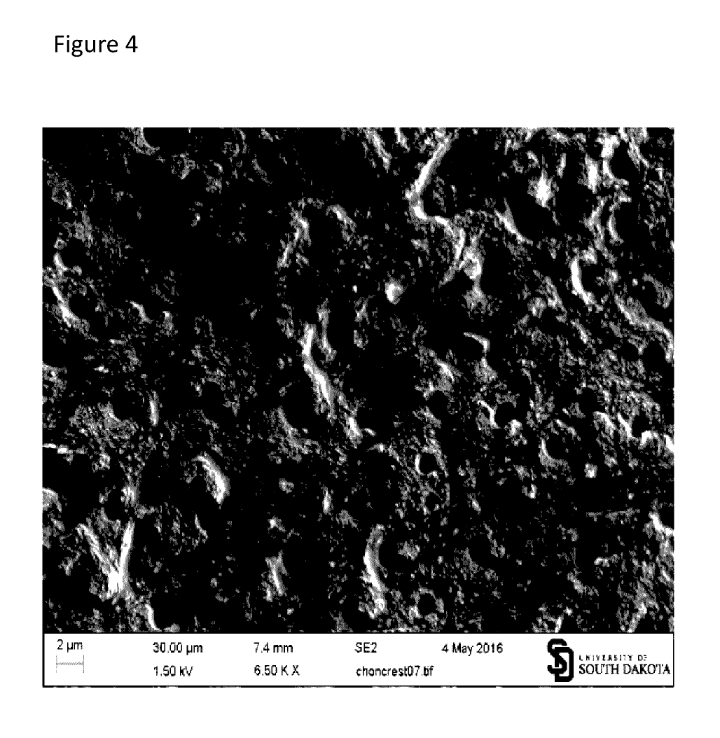

Application 2: An existing toothpaste containing titanium dioxide particles (such as Crest®), that bind to the chondroitin layer due to the complexation of carboxy-groups with titanium, seal the tubules, and thus deliver the desensitizing effect to the tooth. Compare FIG. 3 to FIG. 4.

The adhesion achieved by this method is resistant toward 30 seconds of ultrasound challenge. We have found that the occlusion induced by Sensodyne® does not withstand the ultrasound challenge.

### EXAMPLE 8

Application 1: Mouth rinse containing potassium alginate that binds to the tooth surface due to the presence of carboxy-groups in alginate.

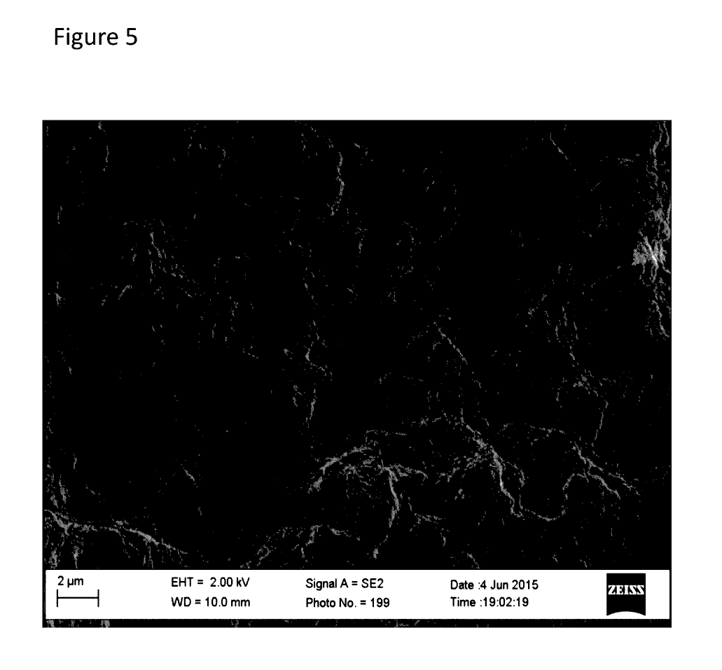

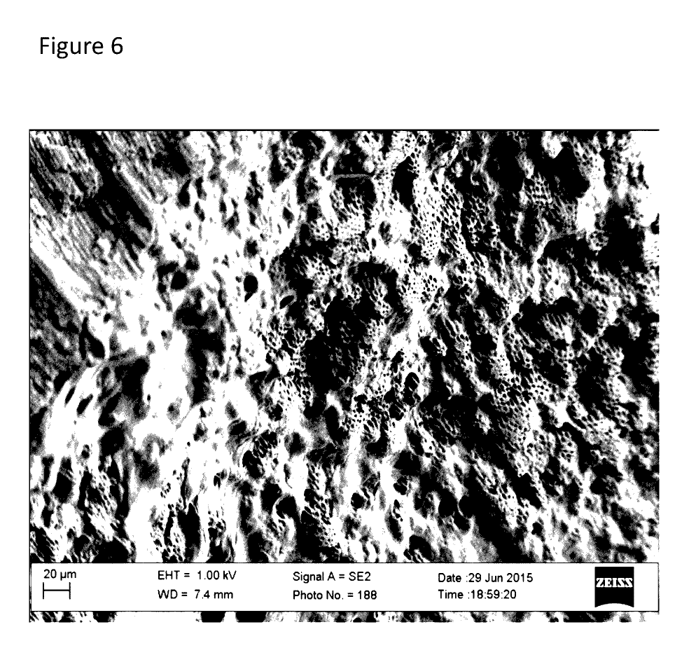

Application 2: Mouth rinse containing calcium ions that cross-links the alginate polymer due to its affinity to calcium-ions, and delivers desensitizing effect to the tooth. Compare FIG. 5 to FIG. 6.

### EXAMPLE 9

Application 1: Mouth rinse containing potassium alginate that binds to the tooth surface due to the presence of carboxy-groups in alginate and delivers the desensitizing material (potassium) to the tooth.

Application 2 is not necessary; the calcium-ions naturally present in saliva cross-link the alginate polymer due to its affinity to calcium ions.

### EXAMPLE 10

Application 1: Consumption of a food containing potassium alginate that binds to the tooth surface due to the presence of carboxy-groups in alginate, and delivers the desensitizing material (potassium) to the tooth.

Application 2: Consumption of a calcium-rich food such as milk. Calcium cations cross-link the alginate inside the dentinal tubules, forming an occlusion matrix loaded with a remineralizing component.

### EXAMPLE 11

Application 1: Wearing dentures, athletic mouth guards, or overnight retainers or mouth guards charged with alginate nanoparticles that bind to the tooth surface due to the presence of carboxy-groups in alginate, and deliver the desensitizing material (potassium) to the tooth.

Application 2: Consumption of a calcium-rich food such as milk. Calcium cations cross-link the alginate inside the dentin tubules, forming an occlusion matrix loaded with a remineralizing component.

EXAMPLE 12

Application 1: Mouth rinse containing antibacterial chitosan (positively charged) that electrostatically interacts with dentin.

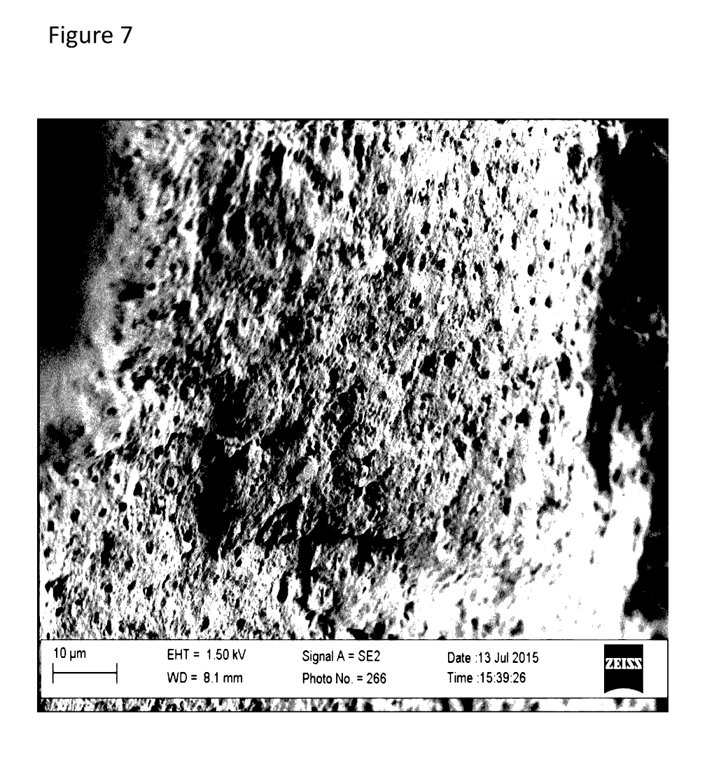

Application 2: Mouth rinse containing citrate-ions that cross-link the already deposited chitosan and seal the tubules delivering the desensitizing effect. Additionally, presence of citrate ion strengthens the bone and provides a site for remineralization due to its affinity to calcium ions. See FIG. 7.

### EXAMPLE 13

Application 1: Chewing gum containing layers of potassium alginate that binds to the tooth surface due to the presence of carboxy-groups in alginate and delivers the desensitizing material (potassium) to the tooth.

Application 2: Additional layers of the same chewing gum containing calcium chondroitinate that is mixed with the alginate by the energy of chewing and cross-linked. The tubules are occluded, and the desensitizing material (potassium) is delivered to the tooth.

### EXAMPLE 14

Application 1: Chewing gum containing microcapsules of potassium alginate that binds to the tooth surface due to the presence of carboxy-groups in alginate and delivers the desensitizing material (potassium) to the tooth.

Application 2: Additional layers of the same chewing gum containing microcapsules of calcium chondroitinate that is mixed with the alginate by the energy of chewing and cross-linked. The tubules are occluded, and the desensitizing material (potassium) is delivered to the tooth.

### EXAMPLE 15

Application 1: Mouth rinse containing calcium ions that are adsorbed on the tooth surface.

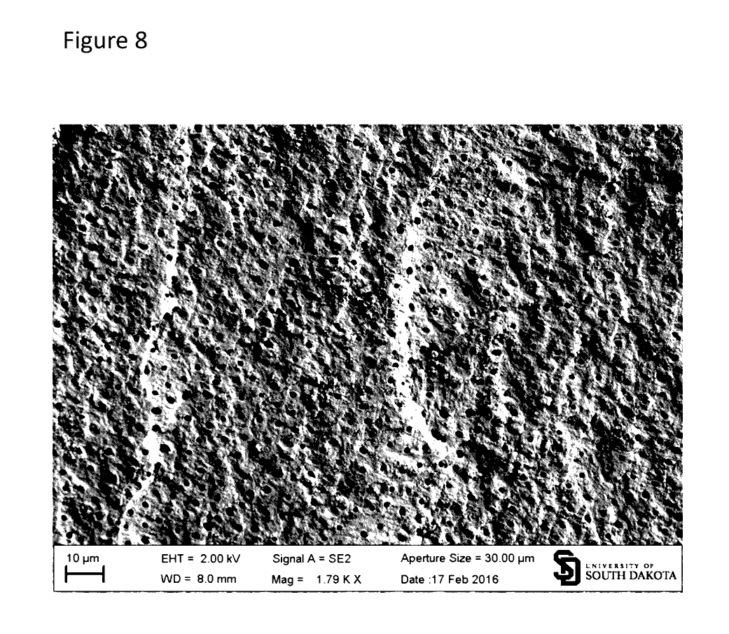

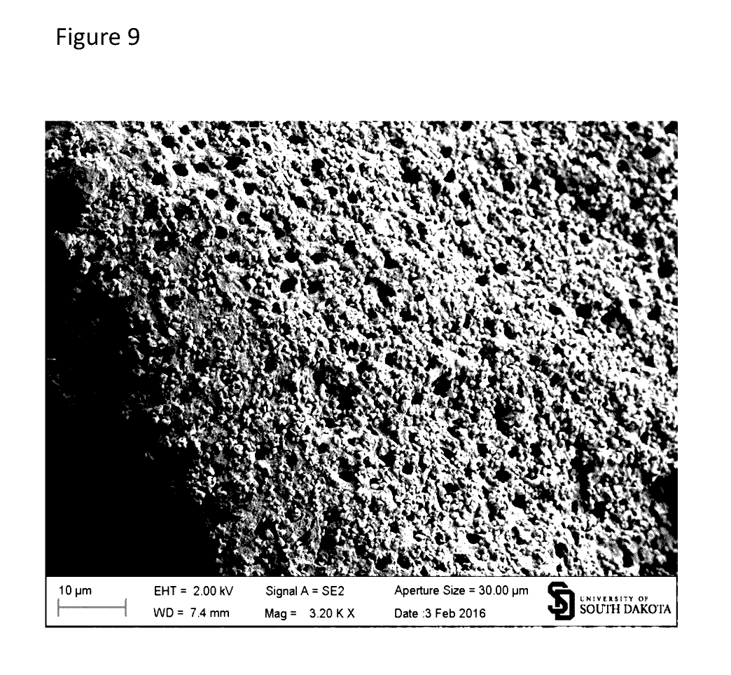

Application 2: A toothpaste containing particles of bentonite that bind to the deposited calcium clue to the complexation of negatively charged groups of bentonite with calcium, occlude the tubules, and thus deliver the desensitizing effect to the tooth. Compare FIG. 8 to FIG. 9.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention. e

### REFERENCES

1. Reynolds, E. Stabilized Calcium Phosphate Complexes. U.S. Pat. No. 8,609,071 B1.
2. Zhang, Q.; Zou, J.; Yang, R.; Zhou, X. Remineralization effects of casein phosphopeptide-amorphous calcium phosphate cre'me on artificial early enamel lesions of primary teeth International Journal of Paediatric Dentistry 2011, 21, 374-381.
3. Gurunathan, D.; Somasundaram, S.; Kumar, S. A. Casein phosphopeptide-amorphous calcium phosphate: a remineralizing agent of enamel. Australian Dental Journal 2012, 57, 404-408.
4. Walker, G. D.; Cai, F.; Shen, P.; Bailey, D. L.; Yuan,Y.; Cochrane, N. J .; Reynolds, C.; Reynolds, E. C. Consumption of milk with added casein phosphopeptideamorphous calcium phosphate remineralizes enamel subsurface lesions in situ Australian Dental Journal 2009, 54, 245-249.
5. Reynolds, E. Treatment for Sensitive Teeth international Patent, 1994, WO 94/00146.
6. Zaidel, L.; Prencipe, M.;Chopra , S. K. Oral Compositions for Treating Tooth Sensitivity and Methods of Use and Manufacture Thereof International Patent, 2010, WO 2010/115041 A2.
7. Diamanti, i.;Koletsi-Kounari, H.; Mamai-Homata, E.; Vougioukiakis, G. In vitro evaluation of fluoride and calcium sodium phosphosilicate toothpastes, on root dentine caries lesions J. Dentistry 2011, 39, 619-628.
8. Narongdej, T.; Sakoolnamarka, R.; Boonroung, Th. The Effectiveness of a Calcium Sodium Phosphosilicate Desensitizer in Reducing Cervical Dentin Hypersensitivity: A Pilot Study JADA, 2010, 141, 8, 995-999.
9. Tschoppe, P.; Zandim, D. L.; Martus, P.; Kielbassa, A. M. Enamel and dentine remineralization by nano-hydroxyapatite toothpastes J. Dentistry 2011, 39,430-437.
10. Rhee S. H.,Tanaka, J. Self-assembly phenomenon of hydroxyapatite nanocrystals on chondroitin sulfate. Journal of Materials Science: Materials in Medicine 2002, 13, 597-600.

What is claimed is:

1. A method of remineralizing teeth, comprising the steps of:
(a) applying to the teeth a first component that attaches to the teeth; and
(b) applying to the teeth treated with the first component a second component that attaches to the first component.

2. The method of claim 1, wherein said first component comprises negatively charged components and said second component comprises positively charged components.

3. The method of claim 2, wherein said first component is selected from the group consisting of bare and functionalized silica particles, chondroitin, hyaluronic acid, alginate, nucleic acids, citrates, and polymer films and wherein said second component is selected from the group consisting of chitosan, polylysine, arginine, calcium cations, proteins, functionalized nano- and micro-particles, and polymer films.

4. The method of claim 1, wherein said first component comprises positively charged components and said second component comprises negatively charged components.

5. The method of claim 4, wherein said first component is selected from the group consisting of chitosan, polylysine, arginine, calcium cations, proteins, functionalized nano- and micro-particles, and polymer films and wherein said second component is selected from the group consisting of bare and functionalized silica particles, chondroitin, hyaluronic acid, alginate, nucleic acids, citrates, and polymer films.

6. The method of claim 1, wherein either said first or second component further comprises nanoparticles, microparticles, polymer films, phospholipids, and small molecules, pristine or modified by remineralizing, desensitizing, antibacterial, fluoride-supplying, and other functional groups.

7. The method of claim 1, wherein said first and second components are contained in different dentifrices, such as toothpaste, mouth rinse, chewing gum, dental floss, toothpicks, dentures, retainers, and the like, or foods, and are deposited on the tooth surface by sequential application of the listed dentifrices or consumption of foods, or combinations of thereof.
